(19) United States
(12) Patent Application Publication
(10) Pub. No.: US 2021/0030698 A1
(43) Pub. Date: Feb. 4, 2021

(54) GEL CONTAINING ZERUMBONE FROM BITTER GINGER (ZINGIBER ZERUMBET) FOR CURATIVE TREATMENT OF DIABETIC ULCERS

(71) Applicants: INSTITUTO NACIONAL DE PESQUISAS DA AMAZÔNIA ? INPA, Manaus - AM (BR); BIOZER DA AMAZÔNIA INDÚSTRIA E COMÉRCIO DE COSMÉTICOS E FITOTERÁPICOS LTDA, Manaus - AM (BR)

(72) Inventors: Carlos Cleomir De Souza PINHEIRO, Manaus - AM (BR); Carlos Danniel Freitas PINHEIRO, Manaus - AM (BR); Gemilson Soares PONTES, Manaus - AM (BR)

(73) Assignees: INSTITUTO NACIONAL DE PESQUISAS DA AMAZÔNIA ? INPA, Manaus - AM (BR); BIOZER DA AMAZÔNIA INDÚSTRIA E COMÉRCIO DE COSMÉTICOS E FITOTERÁPICOS LTDA, Manaus - AM (BR)

(21) Appl. No.: 16/975,504

(22) PCT Filed: Mar. 14, 2019
(86) PCT No.: PCT/BR2019/050084

§ 371 Date: Aug. 25, 2020

(30) Foreign Application Priority Data
Mar. 14, 2018 (BR) 10 2018 005105 9

(51) Int. Cl. A61K 31/122; A61K 9/06; A61K 47/26; A61K 47/32; A61K 9/00

(52) U.S. Cl. CPC A61K 31/122; A61K 9/06; A61K 9/0014; A61K 47/32; A61K 47/26

(57) ABSTRACT

A dermatological pharmaceutical composition in the form of a gel (GZ) for topical use, produced by combining zerumbone (Z) from bitter ginger (Zingiber zerumbet) with carbopol inert hydrogel (G), at a concentration of 0.5 or 1% zerumbone (Z), to be used for the curative treatment of diabetic ulcers, to provide greater healing effectiveness due to the anti-inflammatory, analgesic, healing, antimicrobial, vasodilatory and cytotoxic effects on necrotic cells resulting from the zerumbone.

### FIELD

This patent of invention application refers to an unprecedented GEL CONTAINING ZERUMBONE FROM BITTER GINGER (Zingiber zerumbet) FOR CURATIVE TREATMENT OF DIABETIC ULCERS that describes a pharmaceutical composition of topically applied zerumbone hydrogel employing the therapeutic action of the bitter ginger extract in the treatment of diabetic ulcers, in order to provide greater effectiveness in healing due to its anti-inflammatory, analgesic, healing, antimicrobial, vasodilatory and cytotoxic effects on necrotic cells.

The present invention belongs to the section of human needs; to the health field, more specifically to medicinal preparations; since it is a pharmaceutical composition to be used in the treatment of diabetic ulcers, in order to provide greater effectiveness in healing.

### BACKGROUND

In the current state of the art, there is some prior art that describes the use of zerumbone in pharmaceutical compositions. However, none of them describes a gel in whose composition is zerumbone to be used in the treatment of diabetic ulcers. There is also other prior art that describes compositions developed for healing in diabetics, however, these do not present zerumbone in their formulation.

The document BR102013025449-5, entitled “Uso da zerumbona, formulação dermatológica e/ou cosmética e processo de obtenção” describes the use of zerumbone, dermatological and/or cosmetic formulation comprising the zerumbone and the process of obtaining the dermatological and/or cosmetic formulation. This document proposes the incorporation of bitter ginger extract in cosmetic products, in the case, in solid or liquid soap paste, and its use in the treatment of acnes. Despite employing zerumbone in its composition, it does not mention carbopol gel as a vehicle, which makes it a different product from the proposed invention that is a stable, efficient product and proposes a new use for zerumbone.

The document BRPI0613324, entitled “Composição de aquecimento, composição de resfriamento, kit compreendendo urn creme ou gel de massagem esportiva, kit compreendendo urn gel ou creme de barbear, kit compreendendo urn produto de cuidado pessoal, método para aliviar músculos doloridos, método para preparar uma loção, creme ou spray analgésico tópico ou remédio tópico e método para aumentar o efeito de um material sensibilizador” describes compositions that include (a) a heating agent or a cooling agent and (b) a component containing silicone, providing a method to increase the heating effect of the heating agent and the cooling effect of the cooling agent. This document presents in its composition the regular ginger oil (Zingiber officinale), as one of the options to be used as a heating agent, and the carbopol, as a vehicle in one of its versions. Nevertheless, the proposal does not mention using zerumbone (Zingiber zerumbet) itself, unlike the present patent application, and is used as a local analgesic product.

The document PI0605562-1, entitled “Processo de obtenção do oleo essencial do alecrim para cicatrização de portadores de diabetes de mellitus” describes the process of obtaining the oil from the steam-dragging technique, followed by the addition of anhydrous magnesium sulfate and filtration to obtain pure oil. After obtaining the oil, it is incorporated into a lipophilic ointment based on lanolin in concentrations ranging from 0.5 to 40%. The document presents a product to be used for healing in diabetic patients, as well as the proposal of the present application, however, the active ingredients used are different, which demonstrates the absence of a product on the market with this purpose, based on zerumbone, which presents positive results with regard to the healing process, besides presenting anti-inflammatory, analgesic, antimicrobial, vasodilator and cytotoxic effects to necrotic cells.

From the foregoing, it is observed that none of mentioned prior art anticipates the proposal of this patent application. As much as there are documents that describe the use of zerumbone, none of them is presented for the same purpose described in this application, and although there are compositions with natural extracts for healing in diabetics, they do not present the zerumbone in its formulation. Thus, this proposal is innovative for presenting a new use for zerumbone and for offering the market a different, efficient and stable product.

### SUMMARY

Foot ulcers and amputations are, according to the American Diabetes Society (2004), the main causes of morbidity among people with Diabetes Mellitus; according to Martin et al (2012), the risk of developing these wounds is estimated at 15%. Studies show that the annual population-based incidence can range from 1% to 4.1%, and the prevalence of 4% and 10% (Reiber, 1999). In people affected by diabetes, ulcers are characterized by skin wounds with loss of epithelium, which extend to the dermis or cross it and reach the deepest tissues, and may even reach bones and muscles (Sumbio, 2000) and end up preceding 85% of amputations (Boulton, 2004).

The treatment of chronic ulcers, especially those of venous origin, represents a considerable burden for health services. There is a need for specialized human resources and adjuvant therapies, which are most often adopted for a long time. Additionally, this type of injury often affects the functional capacity of the individual, causing social, psychological and financial impacts.

Venous ulcer represents the most advanced stage of chronic venous insufficiency, with a prevalence of 0.5 to 2% in the world population, being greater than 4% in individuals over 65 years of age. Research developed in recent years has contributed to a significant increase in the availability of products for the healing of these wounds. Dressings based on alginate, papain and other hydrogels have been studied in different concentrations and formulations, as an option in the treatment of ulcers. Evidence on the use of active gels of medicinal plants in the wound healing process, pointed to the deficiency of a pattern of forms, formulations and presentation for the use of the product.

The bitter ginger, Zingiber zerumbet, is a specie originating in Asia, introduced in the Amazon region that has adapted well to the climatic and edaphic conditions of the region. From the rhizomes of this specie is extracted the zerumbone, composed with medicinal properties such as: anti-inflammatory, analgesic, healing, antimicrobial, vasodilator and cytotoxic for necrotic cells.

Based on the properties of zerumbone and the demand for pharmaceutical compositions with standard of forms, formulations and presentation for use, this patent application describes a gel to be used in the treatment of diabetic ulcers. In this sense, this invention proposes the use of the substance zerumbone of bitter ginger in a stable pharmaceutical composition and combined with carbopol inert hydrogel, designed for treatment jointly, in a single formulation and with the benefits that provided by its active component, the zerumbone.

This invention aims to provide a pharmaceutical composition for the treatment of diabetic ulcers, which provides greater effectiveness in healing due to its anti-inflammatory, analgesic, healing, antimicrobial, vasodilator and cytotoxic effects for necrotic cells.

This patent of invention application describes a pharmaceutical composition in dermatological gel formulation, produced from the combination of bitter ginger zerumbone (Zingiber zerumbet) with carbopol inert hydrogel, to be used in the curative treatment of diabetic ulcers, for providing greater effectiveness in healing due to anti-inflammatory, analgesic, healing, antimicrobial, vasodilator and cytotoxic effects for necrotic cells from zerumbone.

In short, this invention presents as main advantages:
provide a topical pharmaceutical composition that presents as an active ingredient the bitter ginger zerumbone (Zingiber zerumbet) in combination with carbopol inert hydrogel;
provide a pharmaceutical composition for the curative treatment of diabetic ulcers with simple obtaining process;
provide a pharmaceutical composition for the stable and fast release curative treatment of diabetic ulcers;
provide a pharmaceutical composition for the curative treatment of diabetic ulcers with a high degree of dissolution;
provide a pharmaceutical composition for the curative treatment of ulcers with greater effectiveness in healing;
Arrange a pharmaceutical composition for the curative treatment of ulcers with anti-inflammatory, analgesic, healing, antimicrobial, vasodilator and cytotoxic effects for necrotic cells.

### DESCRIPTION OF THE FIGURES

The invention will be described in a preferred embodiment, so for better understanding it will be made references to the figures and flowchart:

FIG. 1: Flowchart of the process of obtaining the bitter ginger zerumbone gel;

FIG. 2: Treatment of the patient 1, treated with zerumbone gel in the concentration of 0.5%. A1. Beginning of treatment; A2. After 15 days of treatment; A3: After 45 days of treatment, grade II.

FIG. 3: Treatment of the patient 2, treated with zerumbone gel in the concentration of 0.5%. A1. Beginning of treatment; A2. After 15 days of treatment; A3: After 40 days of treatment, grade II.

FIG. 4: Treatment of the patient 3, treated with zerumbone gel in the concentration of 0.5%. A1. Beginning of treatment; A2. After 15 days of treatment; A3: After 35 days of treatment; A4: After 75 days.

FIG. 5: Treatment of the patient 4, treated with zerumbone gel in the concentration of 1.0%. A1. Beginning of treatment; A2. After 17 days of treatment; A3: After 44 days of treatment.

FIG. 6: Treatment of the patient 5, treated with zerumbone gel in the concentration of 1.0%. A1. Beginning of treatment; A2. After 21 days of treatment; A3: After 44 days of treatment.

FIG. 7: Treatment of the patient 6, treated with zerumbone gel in the concentration of 1.0%. A1. Beginning of treatment; A2. After 22 days of treatment; A3: After 38 days of treatment.

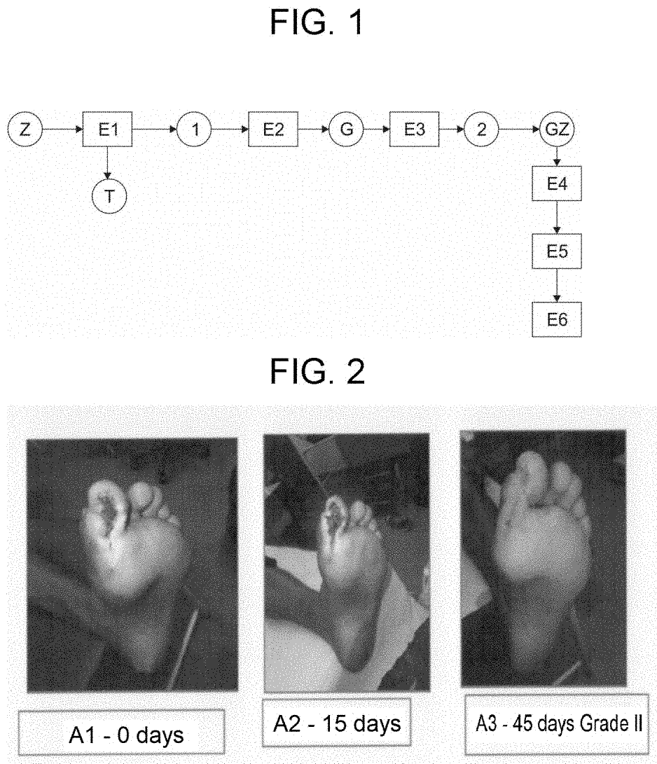

### DETAILED DESCRIPTION

To obtain the zerumbone gel for the curative treatment of ulcers, the zerumbone (Z) obtained from the essential oil of Z. zerumbet (bitter ginger) rhizomes at the concentration of 0.5 to 2% (g/g), was homogenized (E1) with 1% of polysorbate surfactant (T) and, then, the mixture (1) was added (E2) to the inert hydrogel of carbopol (G) at a concentration between 97.5 and 98.5% of the total product, at a temperature between 25 to 30° C. and homogenized (E3) for 15 minutes. After this period, the mixture (2), then in a semi-solid state, will be stabilized, and the product (GZ) can be bottled (E4), then packed (E5) and labeled (E6). The zerumbone gel is valid for 01 year.

The zerumbone (Z) used was obtained from essential oil from the rhizomes of Z zerumbet, which were submitted to the steam-dragging process, using the Clevenger apparatus. The process used to obtain it is known and is part of the current state of the art. Both the zerumbone (Z) obtained from the essential oils, and the essential oils of the rhizomes of Z. zerumbet were characterized physical-chemically, for this, the organoleptic characteristics, pH, density, viscosity, refractive index, optical rotation index, dry residue and solubility (CARDOSO, 2009) were evaluated. Chromatographic analyses of the samples were made, using current techniques of thin layer chromatography and column chromatography (OLIVEIRA, F. et al., 2010). Other physicochemical methods suitable for obtaining the extracts, according to the zerumbone input of the essential oils, fixed or organic extracts, can also be employed; such methods can be cold or hot, namely cold extraction methods: turbolization, maceration and percolation; extraction processes: hydrodistillation, steam-dragging, decoction infusion, reflux or Soxhlet.

Analyses were performed to verify the quality of the carbopol hydrogel (G) used to obtain the zerum bone gel. Thus, as positive control of the tests, samples of calcium alginate hydrogel already found in the market were used. For the physical-chemical analysis of the zerumbone gel (GZ), the organoleptic characteristics, density, pH, viscosity, unit, dry residue and granulometry were evaluated according to the Cosmetic Products Quality Control Guide (ANVISA, 2008).

Foam characterization tests were also performed, for this, a solution was prepared at 5% of the zerumbone gel (GZ), from which 50 ml were poured to a 100 ml beaker and agitated, manually and vertically, 5 consecutive times.

The heights of the formed foam were measured, in milliliters, at the instant the agitation ceased and every 5 minutes for 15 minutes, to evaluate the resistance and general characteristics of the foam.

For stability analysis, studies were conducted according to the Cosmetic Product Stability Guide (ANVISA, 2004). For this, different samples were kept at room temperature, 25±3° C. and in an oven at 40±° C. At 0, 15, 30, 60 and 90 days, they were evaluated for changes in color, odor, pH (measured in 10% solution of samples) and humidity (Karl Fischer method). For the moisture test, the zerumbone gel (GZ) was partially immersed in water for 2 hours at 25° C. After this period, they were removed from the water and exposed to room temperature for 24 hours for drying, after slight scraping on the surface of the tested gels for removal of the dissolved portion and not diluted in water. The zerumbone gels (GZ) were observed for the presence of moisture, viscosity, homogeneity and color, odor, and weighed to evaluate the loss of mass.

The use of bitter ginger (Z. zerumbet) zerumbone gel (GZ), a stable pharmaceutical composition that associates zerumbone (Z) with carbopol hydrogel (G) is used in its topical application for the treatment of diabetic ulcers, providing the benefits of its main active component, zerumbone. The use of the proposed composition brings therapeutic efficacy to the treatment of diabetic ulcers, consequently, it provides greater effectiveness in the curative treatment due to anti-inflammatory, analgesic, healing, antimicrobial, vasodilator and cytotoxic properties for necrotic cells.

Preclinical studies were conducted for the proposed composition in animals in vivo, for this purpose, the use of mice was previously approved by the Ethics Council on Animal Experimentation of INPA. Thus, acute toxicity was evaluated orally in mice of the species Mus musculus of the lethal dose 50% (DL50), according to the method of Morrison et al (1998); Litchfield and Wilcoxon (1949). The used animals were fasting completely (18 hours), after one hour of administration of the extracts, the animals were submitted to muscle tone test (stick test) and exploratory activity test (open field test) and remained under observation for 14 days, by morning time (1 hour). After observation, the animals were sacrificed and then dissected for organ analysis.

The evaluation of primary and cumulative dermal toxicity was carried out in accordance with the Cosmetic Product Safety Assessment Guide (ANVISA, 2003). Primary dermal irritation tests were performed in mice (20 to 30 g) and rats (180 to 300 g) previously shaved. Laboratory animals, as well as some patients in the clinical study had regions tested. Therefore, the dorsal and ear region of the patients and edema of the paw and ear of the laboratory animals had four regions demarcated, two of them superficially scratched with the aid of a sterile needle and the other two remained intact, being used as control. The tested formulations were applied in the four regions through an occlusive patch, composed of fixed sterile gauze; after 04 hours of contact, the product was removed with water. After 24 and 72 hours of application, the presence of erythema and/or edema in the skin of man and animal was evaluated. In the test for cumulative dermal irritation, the applications were made following the same technique, and the samples were reapplied every 24 and 72 hours after the last application for 12 to 14 cycles.

In specific pharmacological tests and preclinical toxicity of acute and subchronic toxicity in mice and rats, it has been reported that essential oil extracts, as well as zerumbone at doses greater than 5000 mg/kg did not cause mortality and showed no toxic effects. In the tests to evaluate the analgesic activity of zerumbone at doses of 250 to 1000 mg/kg, a potent analgesic effect was verified in chemical and thermal models of pain in mice. The zerumbone administered orally and intraperitoneally produced significant, dose-dependent analgesic activity against pain models: acetic acid, formalin and capsaicin. In addition, the zerumbone significantly increased the latency of the animals in the hot plate test pain model (49-54° C.). The results of the effects of anti-inflammatory activity of the substance zerumbone in a model of the edemological inflammatory process (carrageenan, croton oil and arachidonic acid) produced significant and dose-dependent anti-inflammatory effects in inhibiting the inflammatory response of carrageenan in rat paw edema. The effects of the substance zerumbone on barbiturate sleep, using sodium pentobarbital (nembutal), showed that zerumbone altered the induction time (T.I) of barbiturate sleep in recovery time (T.R) by approximately 600 minutes. In the in vitro cytotoxicity tests, zerumbone produced mortality for Artemia salina of CL₅₀—45 ug/ml, showing a positive correlation between antitumor and anti-inflammatory activities. The results showed that the zerumbone presented potent analgesic, anti-inflammatory effects and did not present toxicity in laboratory animals orally.

Physicochemical analyses of zerumbone gel (GZ) revealed the following organoleptic characteristics: bitter spicy taste; colorless to white; as for stability, the product is liquid-solid with odor and characteristics stabled at temperatures below 35° C., and can oxidize in the presence of moisture, light, heat and metals.

The clinical skin dermatological sensitivity test showed that the product, in the applications of concentration of 0.5 to 1% in patients participating in the research group, does not present dermatological sensitivity or skin irritation capable of causing toxic effects significantly when compared to control (negative carbopol) and standard control (calcium alginate hydrogel). Only at the concentrations of bitter ginger gel at the concentration of 1% there was a slight hyperemic alteration in two patients, and the observed effects ceased after a few minutes.

Experimental clinical trials revealed that treatment with zerumbone gel (GZ) at concentrations of 0.5 and 1.0% in the groups of patients with diabetes ulcers was effective and patients showed improvement in the healing process. The groups treated with the gel at the concentration of 0.5% for the periods of 63.33 and 52.44 days obtained between 92.5 and 95% of healing, in addition to the effective inhibition of the inflammatory process and the revitalization and remodeling of the necrotic tissues. The group of patients treated with calcium alginate, for a period of 67.88 days, presented between 30 and 40% healing of the affected areas. Regarding clinical toxicological parameters, none of the groups presented toxic changes or effects related to liver, renal and cardiac functions during treatment.

During the curative tests, patients passed by procedures to clean the wounds with 2% chlorhexidine solution diluted in water, with part of the necrotic tissue being removed with scalpel, without causing damage to the granulation tissue. After hygiene, the zerumbone gel (GZ) was applicated, at the end of the application of the substance to the wound and at the end of the curative process, the wounds were maintained with soaked gauze and subsequently bandaged. After the 48-hour period, a new dressing was made, and the evolution of the curative process of the patients' wounds was observed, which presented fibrin softening and new granulation tissue in healthy areas of the wound, and spontaneous detachment of devitalized tissue with a membranous and sloughed aspect were also verified.

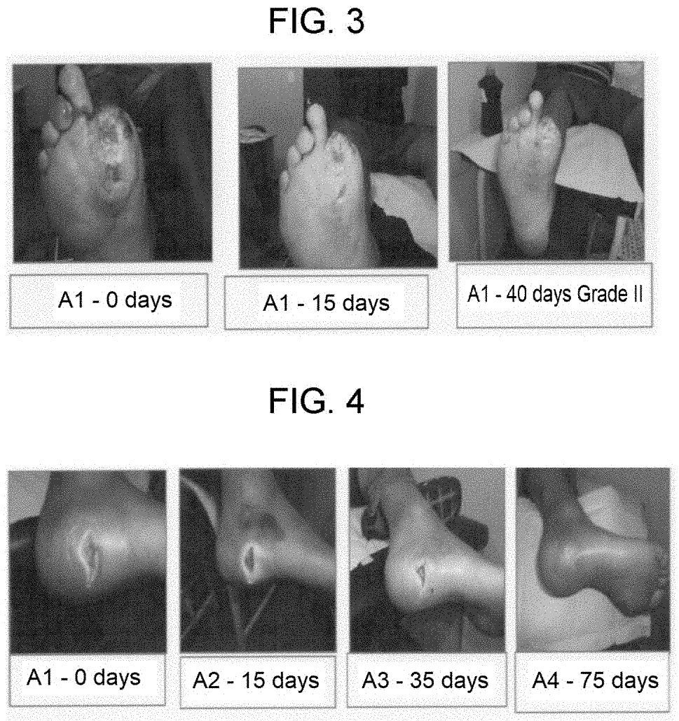

The following clinical examples demonstrate the effectiveness of curative treatment of diabetic ulcers with zerumbone gel (GZ), the subject of this patent application, at a concentration of 0.5%:
1^{st} Case: It was analyzed the Patient 1, male, 62 years old, who had Diabetes Mellitus for 22 years. Venous ulcer in the left foot for 03 years, with wound in the hallucis and plantar region, also characterized by the presence of neuropathy, infection, depth and peripheral vascular disease. At the beginning of treatment with the gel at a concentration of 0.5%, phase 0 of the inflammatory process, the wound presented fetid odor, serous exudate and necrotic tissues with an aspect ranging from whitish to gray. The evolution of treatment was evaluated according to the healing time and period of application of the composition. The results shown in FIG. 2 show that in the period of 15 to 45 days of treatment with zerumbone gel, inflammation was reduced by 95%. During the experiment, the absence of exudate, total reduction of devitalized tissue, increase in fibrinogenic cells (fibrin) with wound retraction and evidence of active granulation, total reduction of necrotic cell levels, in addition to increased evolution of vascular response with migration and activation of leukocytes in systemic reactions of the acute inflammatory process were verified. The results observed are in accordance with the reports of Cotran et al, 2006.
2^{nd} Case: It was analyzed the Patient 2, male, 68 years old, had Diabetes Mellitus for 20 years. Venous ulcers for 05 years, resulting from amputation of the hallucis of the right foot, with two wounds in the plantar region. On day zero of gel treatment at a concentration of 0.5%, the wound presented fetid odor, serous exudate, necrotic and devitalized tissue, fibrin and slough; in addition to partial loss in the ambulation requiring a walking stick. After 15 days of treatment, healing progresses without the presence of any exudate with reduction of devitalized tissue and retraction of the wound and in an active granulation process. After 45 days of treatment, total retraction of the wounds was observed due to the healing process, the evolution of treatment can be observed in FIG. 3.
3^{rd} Case: It was analyzed the Patient 3, male, 62 years old, with type 1 Diabetes Mellitus. Venous ulcers for 05 months in the right foot, outer malleolus region, purulent sloughed-fibrin exudate and devitalized tissue. On day zero of gel treatment at a concentration of 0.5%, the wound presented characteristic odor, purulent exudate and devitalized tissues, besides slough and partial loss in ambulation. After 15 days of treatment, healing evolved without the presence of any exudate with reduction of devitalized tissue and retraction of the wound and in an active moderate granulation process. After 35 days of treatment, keratin was observed in the wound layer and edges with little amount, maintaining granulation and tissue retraction. After 75 days of treatment, there was total retraction of the wound and its integral healing was observed, which can be seen in FIG. 4.

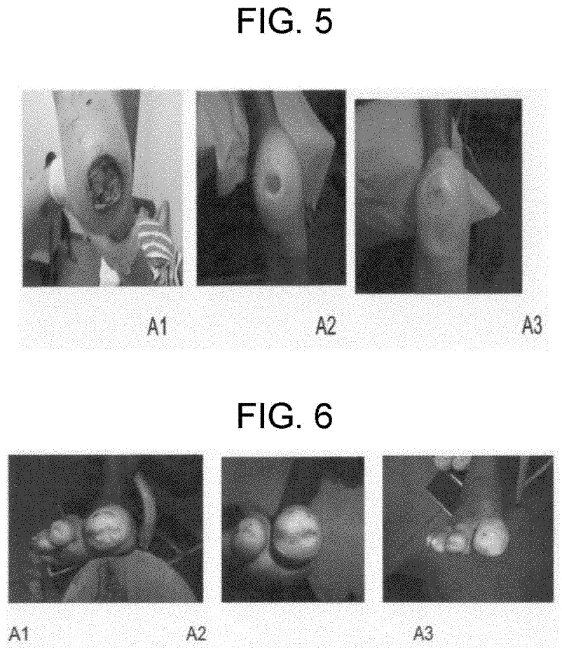

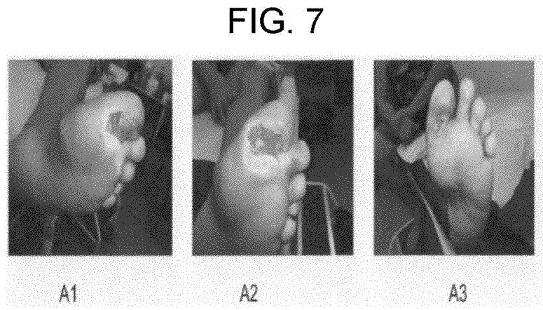

The following clinical examples demonstrate the effectiveness of curative treatment of diabetic ulcers with zerumbone gel (GZ), the subject of this patent application, at a concentration of 1.0%:
1^{st} Case: It was analyzed the Patient 4, male, 50 years old, with venous ulcers for 5 years in the right foot, with wound in the plantar region of the calcaneus; the patient had systemic arterial hypertension, controlled with antihypertensive drugs. On day zero of gel treatment at a concentration of 1.0%, the wound presented fetid odor, serous exudate and necrotic and devitalized tissues, more slough; with partial loss of ambulation. After 15 days of treatment, the wound evolved without the presence of any exudate, and reduction of devitalized tissue and retraction of the wound was observed in an active granulation process. After 35 days of treatment, total retraction of wounds with complete healing process was observed, as can be seen in FIG. 5.
2^{nd} Case: It was analyzed the Patient 5, female, 58 years old with venous ulcers for 16 years in the right foot, with two wounds in the anterior hallucis and external lateral region. On day zero of gel treatment at a concentration of 1.0%, the wound presented fetid odor, bloody exudate, and necrotic and devitalized tissues, more slough; with partial loss of ambulation, requiring walking stick. After 15 days of treatment, the wound evolved without the presence of any exudate, and reduction of devitalized tissue and retraction of the wound was observed in an active granulation process. After 35 days of treatment, total retraction of wounds with complete healing process was observed, as can be seen in FIG. 6.
3^{rd} Case: It was analyzed the Patient 6, female, 62 years old with venous ulcers for 5 years in the hallucis of the right foot, with two wounds in the plantar region and lateral malleolus abscess. The patient had controlled systemic arterial hypertension controlled with antihypertensive drugs. On day zero of gel treatment at a concentration of 1.0%, the wound presented characteristic odor, pain, abscess was drained and received antibiotic therapy, seropurulent exudate and devitalized tissues more slough; with total loss in ambulation, making use of a wheelchair. After antibiotic treatment, the treatment with the gel was restarted. After 22 days of treatment, the wound evolved without the presence of any purulent exudate, and reduction of devitalized tissue and retraction of the wound was observed, in an active and pain-free granulation process. After 38 days of treatment, retraction was observed in more than 70% of the wound due to healing, as can be seen in FIG. 7.

The physical-chemical properties presented by the zerumbone gel (GZ) demonstrates its stability, which is suitable for its storage. During its in vivo release, no incompatibility problems were observed that delayed its release, even at concentrations between 0.5 and 2 mg of zerumbone/g of carbopol hydrogel. The composition still has fast dissolution, its release takes place in less than an hour. In the dissolution tests performed, releases similar to dissolution found in commercial products were observed, thus, they were of the order of 95%.

The pharmaceutical composition of zerumbone and carbopol gel (GZ) is a stable product developed to contain combinations of the active ingredient zerumbone in proportion that allows the control of ulcer healing problems in diabetics, through weekly topical application, for a period between 12 and 15 weeks.

The composition presented is homogeneous and has as differential the anti-inflammatory, healing, vasodilator, analgesic and cytotoxic properties. Therefore, the bitter ginger (Zingiber zerumbet) zerumbone gel (GZ) for the curative treatment of diabetic ulcers is worthy of the patent of invention privilege.

1-5. (canceled)

6. GEL CONTAINING ZERUMBONE FROM BITTER GINGER (Zingiber zerumbet) FOR CURATIVE TREATMENT OF DIABETIC ULCERS, A gel containing zerumbone from bitter ginger (Zingiber zerumbet) for curative treatment of diabetic ulcers, comprising:
Zerumbone (Z) obtained from essential oil from the rhizomes of Z. zerumbet (bitter ginger) at a rate between 0.5 to 2% of the composition;
Polysorbate surfactant (T) at a rate of 1% of the composition; and
Carbopol inert hydrogel (G) at a rate between 97.5 and 98.5% of the composition.

7. The gel containing zerumbone from bitter ginger (Zingiber zerumbet) for curative treatment of diabetic ulcers according to claim 6, wherein the zerumbone gel (GZ) is presented as a gel of topical use, at a concentration of 0.5% of zerumbone (Z) per gram of carbopol hydrogel (G).

8. The gel containing zerumbone from bitter ginger (Zingiber zerumbet) for curative treatment of diabetic ulcers according to claim 6, wherein the zerumbone gel (GZ) is presented as a gel of topical use, at a concentration of 1% of zerumbone (Z) per gram of carbopol hydrogel (G).

9. The gel containing zerumbone from bitter ginger (Zingiber zerumbet) for curative treatment of diabetic ulcers, according to claim 6, wherein the zerumbone gel (GZ) is used as a healing stimulating agent.

10. The gel containing zerumbone from bitter ginger (Zingiber zerumbet) for curative treatment of diabetic ulcers, according to claim 6, wherein the zerumbone gel (GZ) is used in the curative treatment of diabetic ulcers.
